(19) United States
(12) Patent Application Publication
(10) Pub. No.: US 2022/0332763 A1
(43) Pub. Date: Oct. 20, 2022

(54) FUSED POLYPEPTIDE AND USE THEREOF

(71) Applicant: NANJING ANJI BIOLOGICAL TECHNOLOGY CO., LTD., Nanjing (CN)

(72) Inventor: Hanmei XU, Nanjing (CN)

(21) Appl. No.: 17/636,232

(22) PCT Filed: Oct. 26, 2020
(86) PCT No.: PCT/CN2020/123552

§ 371 Date: Feb. 17, 2022

(30) Foreign Application Priority Data
Aug. 27, 2019 (CN) 201910794499.0

(51) Int. Cl. C07K 14/00; A61P 35/00; A61P 19/04

(52) U.S. Cl. CPC C07K 14/001; A61P 35/00; A61P 19/04; A61K 38/00

(57) ABSTRACT

The present invention discloses a fused polypeptide with multifunctional activity and use thereof, relating to the field of biopharmaceuticals. In the fused polypeptide with multifunctional activity, the polypeptide contains the following domains: N-Acetyl-Ser-Asp-Lys-Pro, Ser-Asp-Lys-Pro, Thr-Ser-Leu-Asp-Ala-Ser-Ile-Ile-Trp-Ala-Met-Met-Gln-Asn, and Leu-Ser-Lys-Leu, or domains in which any amino acid in the foregoing domains is mutated. The fused polypeptide can treat various fibrotic diseases including pulmonary fibrosis, hepatic fibrosis, skin fibrosis, renal fibrosis, and myocardial fibrosis, and has activity of inhibiting a plurality of types of human tumor cells.

### TECHNICAL FIELD

The present invention relates to the field of biopharmaceuticals, and in particular, to a fused polypeptide and use thereof.

### BACKGROUND

Fibrosis is a disease that causes a decrease in parenchymal cells of organs and tissues and an increase in fibrillar connective tissues increase. Continuous progression of the disease may lead to structural damage and hypofunction of organs, and eventually failure, which seriously threatens health of patients. Worldwide, fibrosis of tissues and organs is the main cause of disability and death in many diseases.

I. Pulmonary Fibrosis

Pulmonary fibrosis is a lesion mainly caused by uncontrolled repair and regulation and abnormal reconstruction of damaged lung tissues. In this process, oxidative stress caused by a series of abnormal expression of cytokines and growth factors, inflammatory response, vascular proliferation and reconstruction, fibrinolysis disorder, matrix metalloproteinases, external environment, and other factors participates in the pathogenesis of pulmonary fibrosis. This results in major lesions such as epithelial cell deficiency, fibroblast proliferation, and extracellular matrix (ECM) accumulation. A final result is that fibroblasts replace alveolar epithelial cells (AECs) that perform normal functions, leading to the occurrence of fibrosis. The unclear pathogenesis of IPF causes great difficulties to the current treatment, but through experimental research, it can be found that many potential targets are worthy of attention. Because alveoli and AECs are damaged, the body needs to repair the damage, and inflammatory response is also involved. Once the damage repair is excessive or abnormal, the release of some cytokines for chemotaxis and activation of fibroblasts is caused, and the abnormal proliferation of fibroblasts is accompanied by the accumulation of a large number of ECMs, eventually leading to the occurrence of IPF.

A plurality of types of cells, such as pulmonary epithelial cells, endothelial cells, pulmonary inflammatory cells (mainly macrophages), and pulmonary interstitial cells (fibroblasts and myofibroblasts), are involved in the occurrence of fibrosis, and the pulmonary interstitial cells are key effector cells for the occurrence of pulmonary fibrosis. In addition, cytokines secreted by cells, such as transforming growth factor-β (TGF-β), a platelet-derived growth factor (PDGF), a basic fibroblast growth factor (BFGF), a connective tissue growth factor (CTGF), an insulin-like growth factor (IGF), a vascular endothelial growth factor (VEGF), integrin, matrix metalloproteinase (MMP), and an inhibitor (TIMP) thereof, also have a profound impact on the occurrence of pulmonary fibrosis.

The most critical cytokine is TGF-β, which is a multifunctional cell growth factor that can regulate cell proliferation and differentiation. The proliferation of a large number of myofibroblasts and the excessive accumulation of the ECM can be stimulated by directly stimulating the activation of in situ fibroblasts or through endothelial-mesenchymal transition (EnMT) and epithelial-mesenchymal transition (EMT) processes. When TGF-β is continuously activated due to damage, MAPK, EGF, and Wnt/β-catenin signals are cross-activated, leading to the progression of fibrosis. The PDGF, the BFGF, and the VEGF as growth factors can promote the proliferation and differentiation of lung fibroblasts, and affect the progression of pulmonary fibrosis. The MMP/TIMP is a main regulator of the ECM, and the contents of the two play a key role in the balance of the ECM. These cytokines have a more or less influence on the proliferation and activation of lung fibroblasts and the formation of collagen, and therefore reasonable regulation of cytokine expression facilitates the treatment of pulmonary fibrosis.

The polypeptide according to the present invention has a plurality of targets, can inhibit the release of TGF-β1, the proliferation and activation of fibroblasts and the expression of integrin, further inhibit the activation of TGF-β1, inhibit angiogenesis and the expression and release of the VEGF, treat fibrosis in multiple ways, and slow down the process of fibrosis.

2. Hepatic Fibrosis

Hepatic fibrosis is a common pathological change of chronic liver diseases caused by a plurality of causes, characterized by excessive synthesis and degradation reduction of the ECM that is mainly collagen in liver, and the joint control by a plurality of cell signal transduction pathways and a series of signal molecular networks. The activation and proliferation of hepatic stellate cells (HSCs) is an ultimate common way to cause hepatic fibrosis and a central event of hepatic fibrosis. However, a mechanism of occurrence and progression of hepatic fibrosis is very complicated. At present, the research mainly focuses on the activation and transformation of hepatic stellate cells into myofibroblasts and fibroblasts. Possible ways are activation of a TGF-β signal transduction pathway, a PDGF receptor-mediated signal transduction pathway, a TNF-α-mediated signal transduction pathway, cyclooxygenase-2 (COX-2), diffuse ECM, oxidative stress-mediated hepatic fibrosis, or the like.

Hepatic fibrosis is a necessary pathological stage for all kinds of chronic hepatitis to develop into cirrhosis, and is the manifestation of liver injury self-repair. According to a WHO report, there are 20 million cases of hepatitis B virus infection in China, and hepatic fibrosis has occurred to most of these patients. Therefore, how to treat hepatic fibrosis has become an urgent problem to be resolved.

3. Renal Fibrosis

Most chronic renal diseases, such as primary glomerular diseases, chronic pyelonephritis, renal damage caused by systemic diseases (such as lupus nephritis and diabetic nephropathy), and nephropathy (such as Alport syndrome) caused by genetic factors, may lead to renal fibrosis. Renal fibrosis is a pathological process driven by multiple factors, involving inflammation, oxidative stress, functions and signal cascade of a plurality of cytokines, cell apoptosis, proliferation and activation of fibroblasts, transformation of epithelial cells into fibroblasts, and the like.

At present, most drugs for the treatment of renal fibrosis have problems such as high toxicity, low safety, and single pharmacological actions.

Polypeptide drugs have higher druggability than general chemical drugs, have high biological activity, high specificity and relatively weak toxic reaction, and do not easily accumulate in the body. A polypeptide may be designed according to its pathogenesis, is under a multi-target design, and can inhibit the occurrence of renal fibrosis in multiple ways.

4. Skin Fibrosis

Skin fibrosis is excessive scar formation of skin and a result of pathological wound healing response. For many years, scholars at home and abroad have made in-depth research on the mechanism of scar occurrence, progression and regression from multiple angles and levels, but up to now, no clear conclusion is reached on its mechanism, and no effective way for prevention and treatment is available. Relatively consistent views are as follows: \textcircled{1} Fibroblasts are main effector cells of skin fibrosis, which are characterized by excessive cell proliferation and excessive deposition of the extracellular matrix. \textcircled{2} Collagen metabolism disorder is a main biological manifestation of the skin fibrosis. \textcircled{3} A TGF-β1/Smad signaling pathway is closely related to a plurality of physiological and pathological processes such as proliferation, differentiation, migration, apoptosis, and collagen metabolism of fibroblasts. Smads regulate collagen metabolism of fibroblasts bidirectionally according to different types.

The most common method used to treat skin fibrosis is immunosuppressive therapy. The basic principle is that autoimmune causes inflammation of diseases and subsequent tissue damage and fibrosis. Commonly used drugs include methotrexate, cyclophosphamide, and cyclosporine. Although some improvements in immunosuppressive therapy have been observed, concerns about the safety of the drugs and the lack of confirmed clinical data and demonstrable efficacy still exist. Therefore, it is necessary to develop an effective pharmaceutical preparation for the treatment of skin fibrosis, fibrotic skin diseases and pathological scar formation of the skin.

5. Myocardial Fibrosis

Myocardial fibrosis refers to that under the action of various pathogenic factors (such as inflammation, ischemia, and hypoxia), collagen fibers in the normal tissue structure of myocardium are excessively accumulated, the collagen concentration in the heart tissue significantly increases or the collagen composition in the heart tissue changes. Myocardial fibrosis is an important pathological change in the progression of a plurality of cardiovascular diseases, and a final result is myocardial remodeling, stiffness of myocardium, decrease of a ventricular diastolic function, decrease of coronary artery reserves, or even sudden death that may be directly caused. Therefore, prevention and treatment of myocardial fibrosis is of great significance.

### SUMMARY

The Sequence Listing created on Mar. 29, 2022 with a file size of 3.00 KB, and filed herewith in ASCII text file format as the file entitled “Sequence_Listing-G204RAYT0002US.TXT,” is hereby incorporated by reference in its entirety.

### 1. To-be-Resolved Problem

In view of most of existing drugs for treating fibrosis are chemical drugs, and the chemical drugs have problems such as high toxicity, low safety, and single pharmacological actions, the present invention provides a fused polypeptide, which has a good therapeutic effect on lung fibrosis, hepatic fibrosis, renal fibrosis, myocardial fibrosis, and skin fibrosis, and in inhibiting the proliferation of various human tumor cells. The polypeptide according to the present invention contains a plurality of domains, which can target a plurality of targets, and inhibit the occurrence of fibrosis and the proliferation of tumors in multiple ways.

### 2. Technical Solutions

To resolve the foregoing problems, technical solutions adopted by the present invention are as follows:

A fused polypeptide with multifunctional activity, where the polypeptide contains the following domains:

N-Acetyl-Ser-Asp-Lys-Pro (SEQ ID NO: 7), Ser-Asp-Lys-Pro (SEQ ID NO: 7), Thr-Ser-Leu-Asp-Ala-Ser-Ile-Ile-Trp-Ala-Met-Met-Gln-Asn (SEQ ID NO: 8), and Leu-Ser-Lys-Leu (SEQ ID NO: 9), or domains in which any amino acid in the foregoing domains is mutated.

The fused polypeptide is linked by a linker, and the linker is a flexible linker composed of Gly-Gly-Gly-Gly (SEQ ID NO: 10), Ser-Ser-Ser or other amino acids.

Preferably, an amino acid sequence of the polypeptide is as follows:

polypeptide I:
(SEQ ID NO: 1)
Ser-Asp-Lys-Pro-linker-Leu-Ser-Lys-Leu-linker-
Thr-Ser-Leu-Asp-Ala-Ser-Ile-Ile-Trp-Ala-Met-Met-
Gln-Asn;
polypeptide II:
(SEQ ID NO: 2)
Ser-Asp-Lys-Pro-linker-Thr-Ser-Leu-Asp-Ala-Ser-
Ile-Ile-Trp-Ala-Met-Met-Gln-Asn-linker-Leu-Ser-
Lys-Leu;
polypeptide III:
(SEQ ID NO: 3)
Thr-Ser-Leu-Asp-Ala-Ser-Ile-Ile-Trp-Ala-Met-Met-
Gln-Asn-linker-Ser-Asp-Lys-Pro-linker-Leu-Ser-Lys-
Leu;
polypeptide IV:
(SEQ ID NO: 4)
Thr-Ser-Leu-Asp-Ala-Ser-Ile-Ile-Trp-Ala-Met-Met-
Gln-Asn-linker-Leu-Ser-Lys-Leu-linker-Ser-Asp-Lys-
Pro;
polypeptide V:
(SEQ ID NO: 5)
Leu-Ser-Lys-Leu-linker-Ser-Asp-Lys-Pro-linker-Thr-
Ser-Leu-Asp-Ala-Ser-Ile-Ile-Trp-Ala-Met-Met-Gln-
Asn;
and
polypeptide VI:
(SEQ ID NO: 6)
Leu-Ser-Lys-Leu-linker-Thr-Ser-Leu-Asp-Ala-Ser-
Ile-Ile-Trp-Ala-Met-Met-Gln-Asn-linker-Ser-Asp-
Lys-Pro;

where the linker is Gly-Gly-Gly-Gly (SEQ ID NO: 10); and

use of the fused polypeptide in the preparation of anti-pulmonary fibrosis, anti-hepatic fibrosis, anti-renal fibrosis, anti-myocardial fibrosis, and anti-skin fibrosis drugs and antitumor drugs is provided.

The foregoing tumors include human head and neck cancer, brain cancer, thyroid cancer, esophageal cancer, pancreatic cancer, liver cancer, lung cancer, gastric cancer, breast cancer, kidney cancer, colon cancer or rectal cancer, ovarian cancer, cervical cancer, uterine cancer, prostate cancer, melanoma, hemangioma, and sarcoma.

Mechanism of action: The polypeptide according to the present invention has a plurality of targets, and can inhibit the release of TGF-β1, the expression of integrin and angiogenesis, inhibit the activation of fibroblasts in multiple ways, reduce the release of cytokines and the deposition of the extracellular matrix, slow down the foregoing fibrosis process, and further inhibit the proliferation of a plurality of types of human tumor cells.

### 3. Beneficial Effects

Compared with the prior art, the present invention has the following beneficial effects:

(1) The fused polypeptide according to the present invention has excellent anti-fibrosis activity and can be used for treating a plurality of fibrosis diseases, including pulmonary fibrosis, hepatic fibrosis, renal fibrosis, myocardial fibrosis, and skin fibrosis. Components of the fused polypeptide are all natural amino acids, which are easy to synthesize, have no obvious toxic or side effects, and have high safety.

(2) The fused polypeptide according to the present invention can be used for treating pulmonary fibrosis, and in a pulmonary fibrosis model, the polypeptide can significantly improve the structure of the lung, lower a score of pulmonary fibrosis, and improve the survival rate.

(3) The fused polypeptide according to the present invention can be used for treating hepatic fibrosis, and in an in vitro hepatic fibrosis model, the polypeptide can inhibit the proliferation and activation of hepatic stellate cells.

(4) The fused polypeptide according to the present invention can be used for treating renal fibrosis. In a renal fibrosis model, the polypeptide can significantly reduce the expression content of TGF-β1 in renal tissues and significantly improve a situation of renal fibrosis.

(5) The fused polypeptide according to the present invention can be used for treating myocardial fibrosis, and in an in vitro myocardial fibrosis model, the polypeptide can significantly reduce the activation and proliferation of myocardial fibroblasts.

(6) The fused polypeptide according to the present invention can be used for treating skin fibrosis. In a skin fibrosis model, the polypeptide can significantly reduce the expression content of HYP in skin and significantly improve a situation of skin scar hyperplasia.

(7) The fused polypeptide according to the present invention can inhibit the growth of a plurality of types of tumor cells.

(8) The polypeptide according to the present invention is a multi-target drug, and can inhibit the process of fibrosis in multiple ways.

### BRIEF DESCRIPTION OF DRAWINGS

FIG. 1 is a diagram of HE staining of pulmonary fibrosis treated with fused polypeptides I, II, III, IV, V, and VI according to the present invention;

FIG. 2 is a diagram of Masson staining of pulmonary fibrosis treated with the fused polypeptides I, II, III, IV, V and VI according to the present invention;

FIG. 3 shows that fused polypeptides I, II, III, IV, V and VI according to the present invention inhibit the expression content of TGF-β1 in a renal fibrosis model;

FIG. 4 shows that fused polypeptides I, II, III, IV, V and VI according to the present invention inhibit the expression content of HYP in a skin fibrosis model; and

FIG. 5 shows inhibitory effects of the fused polypeptides I, II, III, 1V, V and VI according to the present invention on the growth of different types of tumors.

### DETAILED DESCRIPTION

The polypeptides I, II, III, IV, V, and VI were synthesized by GenScript (Nanjing) Co., Ltd.

### Example 1 Pulmonary Fibrosis Animal Model

Experimental Animals and Materials:

1. Experimental Animals:

Source and strain: clean SD rats, provided by Comparative Medicine Center of Yangzhou University (laboratory animal production license: SCXK (Su) 2012-0004); Laboratory Animal Use License: SYXK (Su) 2012-0035).

Weight: 180-200 g at the time of purchase and 190-210 g at the beginning of modeling.

Gender: Male.

2. Experimental Materials:

| | |
|---|---|
| Bleomycin | Manufacturer: Han Hui Pharmaceutical Co., Ltd. |
| Normal saline | Manufacturer: Anhui Double-Crane Pharmaceutical Co., Ltd. |
| Chloral hydrate | Manufacturer: Sinopharm Chemical Reagent Co., Ltd. |
| BIBF1120 (Nintedanib) | Manufacturer: Jinan Synovel Chemical Co., Ltd. |
| Tissue fixative | Manufacturer: Wuhan servicebio Co., Ltd. |

3. Experimental method:

SD rats were anesthetized by intraperitoneal injection of 1 mL/100 g 4% chloral hydrate. After anesthesia, the rats were fixed and their necks were disinfected by using cotton with 75% alcohol. The skin of the rat neck was longitudinally cut with scissors, and the fascia and muscle were longitudinally bluntly torn with tweezers to expose the trachea. A syringe was inserted into the trachea to inject 5 mg/kg bleomycin, while a blank group was injected with an equal amount of normal saline. Then a rat plate was quickly erected and rotated, the rats' breathing was observed, the neck wound was sterilized after rotation and was sewn, and an amoxicillin anti-inflammatory drug was sprinkled on the suture. After the operation, the rats were put back into a dry and clean cage for resting, waiting was performed for awakening. The rats were awakened after about 1-2 hours, and then fed normally. On the 7^{th} day after modeling, modeling group animals randomly fell into a model group, a Nintedanib positive drug group, polypeptide I, II, III, IV, V, VI dosage groups, and a normal control group, and the groups were administered separately for an administration cycle of 14 days. Living situations of rats were observed every day and their weights were weighed. After administration for 14 days, the SD rats were dissected, the lung tissue was taken, and the right lung tissue was placed in a tissue fixative only for fixation, and HE staining and Masson staining and slice analysis were performed.

4. Experimental Grouping and Dosage Setting

TABLE 1
Experimental grouping and dosage regimen

| | | | Administration | Administration | |
|---|---|---|---|---|---|
| Group | Drug | Dosage | mode | frequency | Quantity |
| Blank group | Normal saline | 0.5 mL/200 g | Subcutaneous injection | Twice a day | 10 |
| Model group | Normal saline | 0.5 mL/200 g | Subcutaneous injection | Twice a day | 10 |
| Positive drug | Nintedanib | 25 mg/kg | Intragastric administration | Once a day | 10 |
| Test drug (1) | Polypeptide I | 10 mg/kg | Subcutaneous injection | Twice a day | 10 |
| Test drug (2) | Polypeptide II | 10 mg/kg | Subcutaneous injection | Twice a day | 10 |
| Test drug (3) | Polypeptide III | 10 mg/kg | Subcutaneous injection | Twice a day | 10 |
| Test drug (4) | Polypeptide IV | 10 mg/kg | Subcutaneous injection | Twice a day | 10 |
| Test drug (5) | Polypeptide V | 10 mg/kg | Subcutaneous injection | Twice a day | 10 |
| Test drug (6) | Polypeptide VI | 10 mg/kg | Subcutaneous injection | Twice a day | 10 |

4. Experimental Results

(1) Impact of a Polypeptide on the Survival Rate of SD Rats Induced by Bleomycin

As shown in Table 2, compared with the survival rate (50%) of SD rats in the model group, the survival rate of SD rats in each test dnig group was higher than that of the model group, and each test drug could significantly increase the survival rate of SD rats, and the survival rate of the polypeptide I group was equivalent to that of the positive drug group.

TABLE 2
Impact of a polypeptide on survival rate (%) of SD
rats with bleomycin-induced pulmonary fibrosis

| | | Number | | |
|---|---|---|---|---|
| | | of animals | Number | |
| | Dosage | at the | of animals | Survival |
| Group | (mg/kg) | beginning | at the end | rate (%) |
| Blank group | — | 10 | 10 | 100 |
| Model group | — | 10 | 5 | 50 |
| Positive drug group | 10 | 10 | 9 | 90 |
| Polypeptide I | 10 | 10 | 9 | 90 |
| Polypeptide II | 10 | 10 | 8 | 80 |
| Polypeptide III | 10 | 10 | 8 | 80 |
| Polypeptide IV | 10 | 10 | 8 | 80 |
| Polypeptide V | 10 | 10 | 7 | 70 |
| Polypeptide VI | 10 | 10 | 7 | 70 |

2. Pathological Analysis of a Polypeptide on Bleomycin-Induced Pulmonary Fibrosis in SD Rats

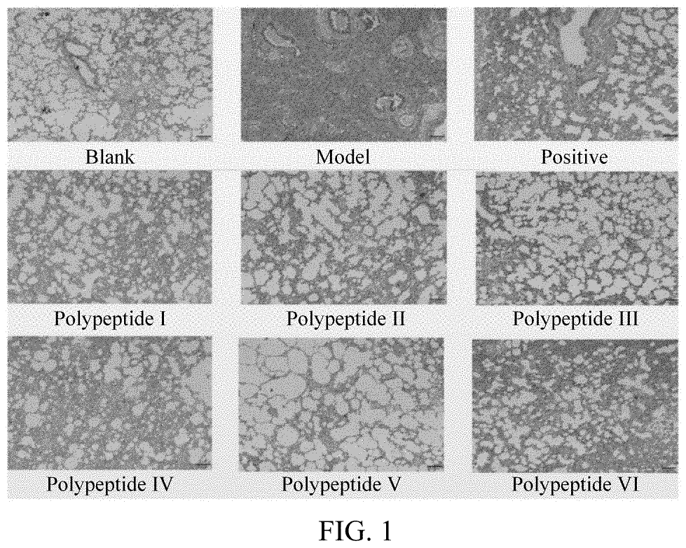

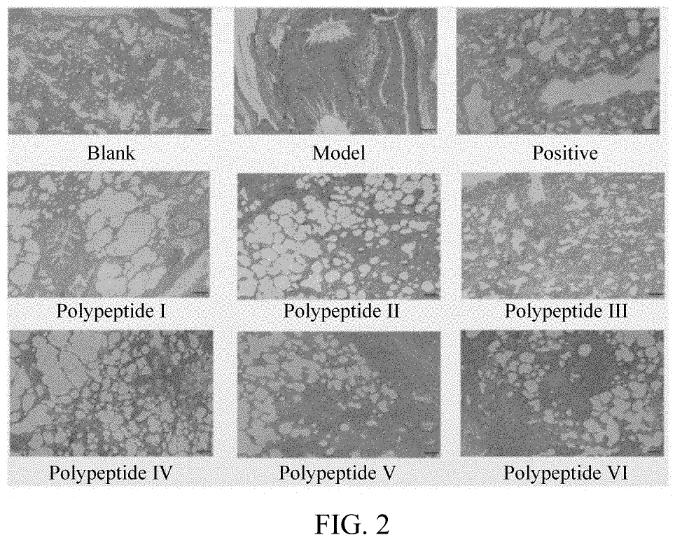

Research results showed that a pulmonary fibrosis model in SD rats was successfully established in this study. Main manifestations of lung tissue lesions are fibroblast proliferation and collagen fiber formation in the alveolar wall and mesenchyme around intrapulmonary bronchi and vascular branches. Masson staining showed blue-green staining reaction, and inflammatory cell infiltration, congestion in the alveolar wall, cell degeneration disorder and other lesions occurred. After administration, the degree of pulmonary fibrosis and other lesions were less than those in the model group. See FIG. 1 and FIG. 2 for HE staining and Masson staining.

### Example 2 In Vitro Hepatic Fibrosis Model

1. Experimental Method

The inhibitory effect of a polypeptide on LX-2 hepatic stellate cells was detected by MTT assay. Cells were cultured in a 1640 medium containing 10% of FBS, the cytoplasm was made into 4×10⁵/mL cell suspension, and 100 μL per well was inoculated into a 96-well plate. After the cells adhered to the wall, the medium was replaced with a serum-free 1640 medium, and the serum-free medium was discarded after 24 hours. The cells were cultured with different polypeptides of 1 μmol/L, and 5 multiple wells were set for each concentration. After 12, 24 and 48 hours separately, 10 μL of MTT was added to each well. After 4 hours, MTT was sucked out, and 150 μL of DMSO was added to each well. After reaction for 5 min, an OD value was measured at 570 nm by a microplate reader.

2. Experimental Results

At 24 hours and 48 hours, polypeptides I, II, III, IV, V and VI could inhibit the proliferation of cardiac fibroblasts of rats at 1 μmol/L. The results are shown in Table 3:

TABLE 3
Impact of a polypeptide on the proliferation of LX-2 hepatic stellate cells

| | Optical density values at different time points | | |
|---|---|---|---|
| Group | 12 h | 24 h | 48 h |
| Blank group | 0.456 ± 0.012 | 0.548 ± 0.01 | 0.812 ± 0.016 |
| Polypeptide I | 0.452 ± 0.008 | 0.542 ± 0.03 | 0.680 ± 0.014*** |
| (1 μmol/L) | | | |
| Polypeptide II | 0.463 ± 0.012 | 0.394 ± 0.005*** | 0.578 ± 0.005*** |
| (1 μmol/L) | | | |
| Polypeptide III | 0.455 ± 0.002 | 0.435 ± 0.013** | 0.642 ± 0.018* |
| (1 μmol/L) | | | |
| Polypeptide IV | 0.478 ± 0.018 | 0.472 ± 0.03** | 0.580 ± 0.012*** |
| (1 μmol/L) | | | |
| Polypeptide V | 0.462 ± 0.004 | 0.477 ± 0.015** | 0.618 ± 0.015*** |
| (1 μmol/L) | | | |
| Polypeptide VI | 0.453 ± 0.021 | 0.502 ± 0.013* | 0.652 ± 0.018* |
| (1 μmol/L) | | | |

***P < 0.001,
**P < 0.01,
*P < 0.05 VS control.

### Example 3 Establishment of a Renal Fibrosis Model

1. Experimental Animals

Clean grade male SD rats, purchased from Nanjing Qinglong Mountain Animal Farm, and weighed 180-200 g at the time of purchase, 190-210 g at the beginning of modeling, and 180-200 g at the beginning of administration.

2. Experimental Materials:

Normal saline Manufacturer: Anhui Double-Crane Pharmaceutical Co., Ltd.

Rat TGF-β1 ELISA kit Manufacturer: Tianjin Annuo Ruikang Biotechnology Co., Ltd.

3. Experimental Method

A renal fibrosis animal model was established. SD rats were anesthetized with 4% chloral hydrate, injected with 1 mL/100 g intraperitoneally, fixed to an operation board, and sterilized in an operation area for later use. The abdominal cavity was cut open about 3-4 mm to the left of the ventrimeson, left kidney ureter was separated in an operation group, the ureter was ligated and separated close to the ureter near the lower pole of the inferior pole of kidney, and the ureter was cut short between two ligations after the double ligations. Muscular layers and abdominal walls were sewed layer by layer, the suture was disinfected with alcohol. After SD rats woke up, the rats were put into a cage for feeding. In the blank group, ureter was not ligated, and other steps were the same.

Then, the animals fell into a blank group, a model group, and polypeptide administration groups, with 10 animals in each group, and the administration was started on the second day after the operation, twice a day for 14 days. After administration for 14 days, blood was taken and supernatant was taken to detect the content of TGF-β1 in serum.

4. Experimental Grouping and Dosage Setting

TABLE 4
Experimental grouping and dosage regimen

| | | | Administration | Administration | |
|---|---|---|---|---|---|
| Group | Drug | Dosage | mode | frequency | Quantity |
| Blank group | Normal saline | 0.5 mL/200 g | Subcutaneous injection | Once a day | 10 |
| Model group | Normal saline | 0.5 mL/200 g | Subcutaneous injection | Once a day | 10 |
| Test drug (1) | Polypeptide I | 7.5 mg/kg | Subcutaneous injection | Twice a day | 10 |
| Test drug (2) | Polypeptide II | 7.5 mg/kg | Subcutaneous injection | Twice a day | 10 |
| Test drug (3) | Polypeptide III | 7.5 mg/kg | Subcutaneous injection | Twice a day | 10 |
| Test drug (4) | Polypeptide IV | 7.5 mg/kg | Subcutaneous injection | Twice a day | 10 |
| Test drug (5) | Polypeptide V | 7.5 mg/kg | Subcutaneous injection | Twice a day | 10 |
| Test drug (6) | Polypeptide VI | 7.5 mg/kg | Subcutaneous injection | Twice a day | 10 |

5. Experimental Results

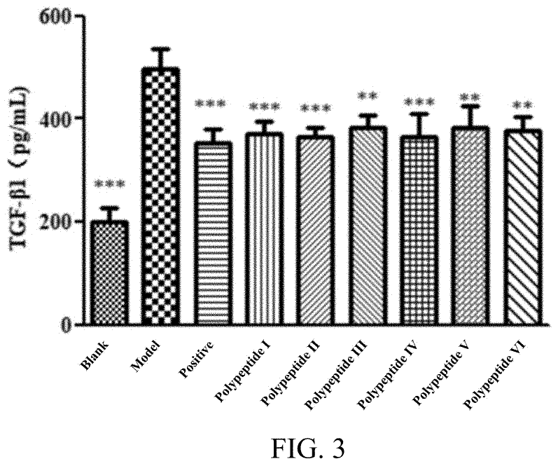

(1) Impact of a polypeptide on the content of TGF-β1 in serum of SD rats with renal fibrosis TGF-β1 is the most important fibrogenic factor. In renal fibrosis, the expression of TGF-β1 was significantly increased. The result is shown in FIG. 3, and there was a highly significant difference between the model group and the blank group (***p<0.001). After administration, all groups could significantly reduce the content of TGF-β1 in serum, and the polypeptide I group, the polypeptide II group and the polypeptide IV group were highly significantly different from the model group (***P<0.001), and the polypeptide III group, the polypeptide V group and the polypeptide VI group were highly significantly different from the model group (**P<0.01).

### Example 4 Establishment of a Myocardial Fibrosis Model

1. Experimental Method

The inhibitory effect of a polypeptide on cardiac fibroblasts of rats was detected by MTT assay. Cells were cultured in a DMEM medium containing 10% of FBS, the cytoplasm was made into 1×10⁵/mL cell suspension, and 100 μL per well was inoculated into a 96-well plate. After the cells adhered to the wall, the medium was replaced with a serum-free DMEM medium, and the serum-free medium was discarded after 24 hours. The cells were cultured with different polypeptides of 1 μmol/L, and 5 multiple wells were set for each concentration. After 12, 24 and 48 hours separately, 10 μL of MTT was added to each well. After 4 hours, MTT was sucked out, and 150 μL of DMSO was added to each well. After reaction for 5 min, an OD value was measured at 570 nm by a microplate reader.

2. Experimental Results

At 24 hours and 48 hours, polypeptides I, II, III, IV, V, and VI could inhibit the proliferation of cardiac fibroblasts of rats at 1 μmol/L. The results are shown in Table 5.

TABLE 5
Impact of a polypeptide on the proliferation of cardiac fibroblasts of rats

| | Optical density values at different time points | | |
|---|---|---|---|
| Group | 12 h | 24 h | 48 h |
| Blank group | 0.353 ± 0.001 | 0.464 ± 0.018 | 0.896 ± 0.001 |
| Polypeptide I | 0.362 ± 0.006 | 0.402 ± 0.002* | 0.678 ± 0.002** |
| (1 μmol/L) | | | |
| Polypeptide II | 0.352 ± 0.004 | 0.367 ± 0.016*** | 0.568 ± 0.013*** |
| (1 μmol/L) | | | |
| Polypeptide III | 0.349 ± 0.012 | 0.413 ± 0.003* | 0.612 ± 0.018** |
| (1 μmol/L) | | | |
| Polypeptide IV | 0.362 ± 0.015 | 0.392 ± 0.008*** | 0.583 ± 0.012*** |
| (1 μmol/L) | | | |
| Polypeptide V | 0.357 ± 0.024 | 0.397 ± 0.015*** | 0.588 ± 0.019*** |
| (1 μmol/L) | | | |
| Polypeptide VI | 0.340 ± 0.012 | 0.412 ± 0.005* | 0.622 ± 0.007* |
| (1 μmol/L) | | | |

***P < 0.001,
**P < 0.01,
*P < 0.05 VS control.

### Example 6 Establishment of a Skin Fibrosis Model

1. Experimental Animals

Male C57/BL black mice aged 6-8 weeks, purchased from Nanjing Qinglong Mountain Animal Fai in.

2. Experimental Materials

| | |
|---|---|
| Bleomycin | Manufacturer: Han Hui Pharmaceutical Co., Ltd. |
| Normal saline | Manufacturer: Anhui Double-Crane Pharmaceutical Co., Ltd. |
| Rat TGF-β1 ELISA kit | Manufacturer: Tianjin Annuo Ruikang Biotechnology Co., Ltd. |
| Alkaline HYP kit | Manufacturer: Nanjing Jiancheng Bioengineering Institute |

3. Modeling Method

Bleomycin (10 μg/mL) was injected subcutaneously every day for 28 days to form skin fibrosis. During the modeling period, the administration groups were given polypeptide drugs twice a day for treatment. After modeling, the mice were killed on the next day, and the skin tissue of the mouse back was taken to detect the content of HYP in the skin tissue.

4. Experimental Grouping and Dosage Regimen

TABLE 6
Experimental grouping and dosage regimen

| | | | Administration | Administration | |
|---|---|---|---|---|---|
| Group | Drug | Dosage | mode | frequency | Quantity |
| Blank group | Normal saline | 0.2 mL | Subcutaneous injection | Twice a day | 10 |
| Model group | Normal saline | 0.2 mL | Subcutaneous injection | Twice a day | 10 |
| Test drug (1) | Polypeptide I | 10 mg/kg | Subcutaneous injection | Twice a day | 10 |
| Test drug (2) | Polypeptide II | 10 mg/kg | Subcutaneous injection | Twice a day | 10 |
| Test drug (3) | Polypeptide III | 10 mg/kg | Subcutaneous injection | Twice a day | 10 |
| Test drug (4) | Polypeptide IV | 10 mg/kg | Subcutaneous injection | Twice a day | 10 |
| Test drug (5) | Polypeptide V | 10 mg/kg | Subcutaneous injection | Twice a day | 10 |
| Test drug (6) | Polypeptide VI | 10 mg/kg | Subcutaneous injection | Twice a day | 10 |

5. Experimental Results

(1) Expression of HYP Content in the Skin Tissue of Each Group of Mice

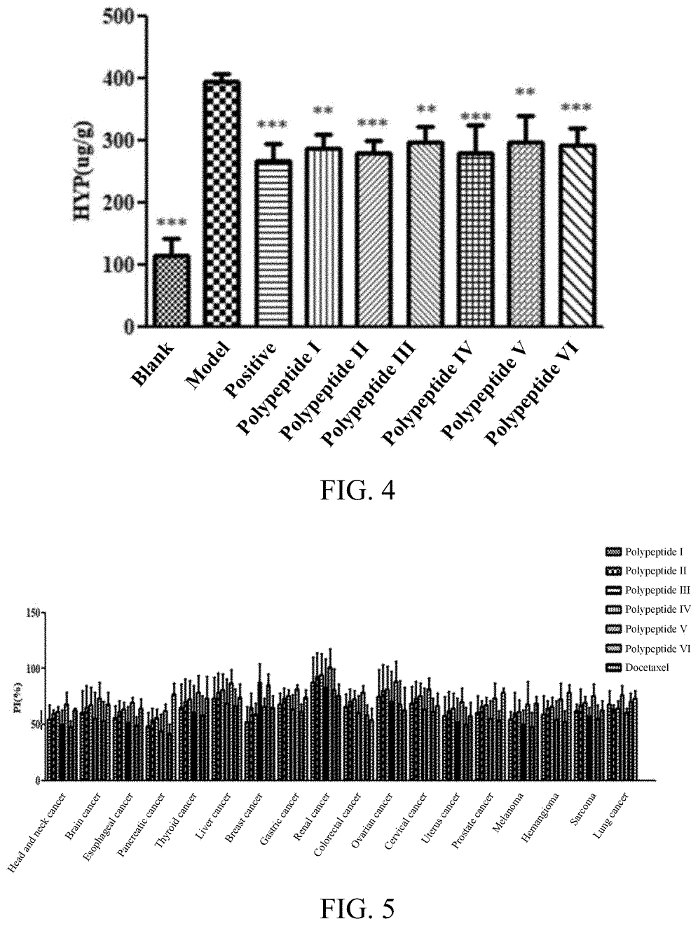

The content of hydroxyproline in the skin tissue of the mouse back was detected. As the characteristic protein of collagen, hydroxyproline can reflect the content of collagen in the skin tissue from the side. As shown in FIG. 4, each polypeptide group could reduce the expression of HYP in the skin tissue. The polypeptide II group, the polypeptide IV group and the polypeptide VI group could significantly reduce the expression of HYP in the lung tissue, and were highly significantly different from the model group (***P<0.001). The polypeptide I group, the polypeptide III group and the polypeptide V group could reduce the content of HYP in the lung tissue of SD rats, and were highly significantly different from the model group (*P<0.05).

### Example 7 Inhibitory Effect of a Polypeptide According to the Present Invention on the Growth of Tumor Cells from a Plurality of Sources Detected by Using MITT Assay

A plurality of types of human tumor cells were cultured in a 5% CO₂ incubator at 37° C. and digested with trypsin when the density was 90% or above. The cells were resuspended in a culture solution and counted, and the cell concentration was adjusted to 2×10⁴ cells/mL. The cell suspension was inoculated into a 96-well plate with 100 μL per well, and then cultured overnight in a 5% CO₂ incubator at 37° C. After the cells completely adhered to the wall, each polypeptide according to the present invention was added as an administration group, and the culture solution without any drug was used as a blank control group. The solutions were diluted to 1 μmol/L by using a diluent. Each diluent was separately added to the 96-well plate with 100 per well, and the cells continued to be cultured in a 5% CO₂ incubator for 48 hours at 37° C. Then 20 μL of MTT was added, and the cells continued to be cultured for 4 hours. The medium was sucked, and 100 μL of DMSO was added to each well for dissolution. Absorbance was measured by a microplate reader at a detection wavelength of 570 nm and a reference wavelength of 630 nm, and the growth inhibition rate was calculated. The formula was as follows: tumor growth inhibition rate (%)=(1−absorbance of the administration group/absorbance of the blank group)*100%. The experiment was repeated independently for 3 times. Experimental results were expressed by mean±standard deviation, and the tumor growth inhibition rate of the blank group was 0. Results in Table 8 showed that the polypeptide according to the present invention had a significant inhibitory effect on the growth of a plurality of types of tumors (FIG. 5).

TABLE 7
Inhibitory effect (%) of a polypeptide according to the present invention on the growth of a plurality of types of tumors detected by MTT assay

| | Polypeptide | Polypeptide | Polypeptide | Polypeptide | Polypeptide | Polypeptide | |
|---|---|---|---|---|---|---|---|
| Tumor type | I | II | III | IV | V | VI | Docetaxel |
| Head and neck cancer | 54.48 ± 12.59 | 59.48 ± 2.98 | 61.48 ± 3.99 | 49.68 ± 13.16 | 67.68 ± 10.66 | 47.48 ± 5.81 | 62.48 ± 2.12 |
| Brain cancer | 60.13 ± 20.12 | 65.13 ± 19.36 | 67.13 ± 16.15 | 55.33 ± 23.49 | 73.33 ± 14.34 | 53.13 ± 16.94 | 68.13 ± 10.26 |
| Esophageal cancer | 56.33 ± 10.53 | 61.33 ± 9.75 | 63.33 ± 6.54 | 51.53 ± 13.88 | 69.53 ± 4.75 | 49.33 ± 7.35 | 64.39 ± 8.06 |
| Pancreatic cancer | 48.79 ± 11.54 | 53.79 ± 10.76 | 55.79 ± 7.55 | 43.99 ± 14.89 | 61.99 ± 5.76 | 41.79 ± 8.36 | 76.74 ± 10.09 |
| Thyroid cancer | 65.26 ± 20.71 | 70.26 ± 19.93 | 72.26 ± 16.72 | 60.46 ± 24.06 | 78.46 ± 14.93 | 58.26 ± 17.53 | 73.21 ± 19.26 |
| Liver cancer | 73.42 ± 18.21 | 78.42 ± 17.43 | 80.42 ± 14.22 | 68.62 ± 21.56 | 86.62 ± 12.43 | 66.42 ± 15.03 | 74.22 ± 11.71 |
| Breast cancer | 52.15 ± 13.36 | 65.35 ± 12.58 | 59.15 ± 9.37 | 87.38 ± 16.71 | 65.38 ± 7.58 | 85.18 ± 10.18 | 65.12 ± 10.66 |
| Gastric cancer | 68.14 ± 9.86 | 73.14 ± 9.08 | 75.14 ± 5.87 | 63.34 ± 13.21 | 81.34 ± 4.08 | 61.14 ± 6.68 | 74.16 ± 6.38 |
| Kidney cancer | 87.48 ± 22.39 | 92.48 ± 21.61 | 94.48 ± 18.4 | 82.68 ± 25.74 | 85.68 ± 16.61 | 80.48 ± 19.21 | 75.48 ± 10.23 |
| Colorectal cancer | 65.55 ± 11.54 | 70.55 ± 10.76 | 72.55 ± 7.55 | 60.75 ± 14.89 | 78.7 ± 5.76 | 58.55 ± 8.36 | 53.55 ± 10.41 |
| Ovarian cancer | 74.75 ± 24.12 | 79.75 ± 23.34 | 81.75 ± 20.13 | 69.95 ± 27.47 | 87.95 ± 18.34 | 67.75 ± 20.94 | 62.75 ± 20.23 |
| Cervical cancer | 68.47 ± 15.31 | 73.47 ± 14.53 | 75.47 ± 11.32 | 63.67 ± 18.66 | 81.67 ± 9.53 | 61.47 ± 12.13 | 66.56 ± 11.31 |
| Uterus cancer | 57.2 ± 17.76 | 62.2 ± 16.98 | 64.2 ± 13.77 | 52.4 ± 21.11 | 70.4 ± 11.98 | 50.2 ± 14.58 | 57.24 ± 12.28 |
| Prostate cancer | 60.4 ± 15.12 | 65.4 ± 5.53 | 67.4 ± 6.54 | 55.6 ± 15.71 | 73.6 ± 13.21 | 53.4 ± 8.36 | 78.4 ± 4.21 |
| Melanoma | 54.48 ± 6.54 | 59.48 ± 19.12 | 61.48 ± 10.31 | 49.68 ± 12.76 | 67.68 ± 20.32 | 47.48 ± 13.32 | 68.42 ± 6.23 |
| Hemangioma | 58.98 ± 16.59 | 63.98 ± 6.98 | 65.98 ± 7.99 | 54.18 ± 17.16 | 72.18 ± 14.66 | 51.98 ± 9.81 | 78.76 ± 6.16 |
| Sarcoma | 62.15 ± 5.54 | 67.15 ± 14.12 | 69.15 ± 5.31 | 57.35 ± 7.76 | 75.35 ± 10.86 | 55.15 ± 12.32 | 62.51 ± 8.75 |
| Lung cancer | 68.15 ± 12.21 | 68.15 ± 12.21 | 63.42 ± 3.51 | 64.57 ± 6.77 | 76.45 ± 8.06 | 60.87 ± 3.12 | 73.32 ± 7.03 |

1. A fused polypeptide with multifunctional activity, wherein the polypeptide comprises the following domains:
N-Acetyl-Ser-Asp-Lys-Pro (SEQ ID NO: 7), Ser-Asp-Lys-Pro (SEQ ID NO: 7), Thr-Ser-Leu-Asp-Ala-Ser-Ile-Ile-Trp-Ala-Met-Met-Gln-Asn (SEQ ID NO: 8), and Leu-Ser-Lys-Leu (SEQ ID NO: 9), or domains in which any amino acid in the foregoing domains is mutated.

2. The fused polypeptide with multifunctional activity according to claim 1, wherein the fused polypeptide is linked by a linker, and the linker is a flexible linker composed of Gly-Gly-Gly-Gly (SEQ ID NO: 10), Ser-Ser-Ser or other amino acids.

3. The fused polypeptide with multifunctional activity according to claim 2, wherein an amino acid sequence of the fused polypeptide is the following sequence or a sequence with 80% homology therewith:
polypeptide I:
(SEQ ID NO: 1)
N-Acetyl-Ser-Asp-Lys-Pro-Gly-Gly-Gly-Gly-Leu-Ser-
Lys-Leu-Gly-Gly-Gly-Gly-Thr-Ser-Leu-Asp-Ala-Ser-
Ile-Ile-Trp-Ala-Met-Met-Gln-Asn;
polypeptide II:
(SEQ ID NO: 2)
N-Acetyl-Ser-Asp-Lys-Pro-Gly-Gly-Gly-Gly-Thr-Ser-
Leu-Asp-Ala-Ser-Ile-Ile-Trp-Ala-Met-Met-Gln-Asn-
Gly-Gly-Gly-Gly-Leu-Ser-Lys-Leu;
polypeptide III:
(SEQ ID NO: 3)
Thr-Ser-Leu-Asp-Ala-Ser-Ile-Ile-Trp-Ala-Met-Met-
Gln-Asn-Gly-Gly-Gly-Gly-Ser-Asp-Lys-Pro-Gly-Gly-
Gly-Gly-Leu-Ser-Lys-Leu;
polypeptide IV:
(SEQ ID NO: 4)
Thr-Ser-Leu-Asp-Ala-Ser-Ile-Ile-Trp-Ala-Met-Met-
Gln-Asn-Gly-Gly-Gly-Gly-Leu-Ser-Lys-Leu-Gly-Gly-
Gly-Gly-Ser-Asp-Lys-Pro;
polypeptide V:
(SEQ ID NO: 5)
Leu-Ser-Lys-Leu-Gly-Gly-Gly-Gly-Ser-Asp-Lys-Pro-
Gly-Gly-Gly-Gly-Thr-Ser-Leu-Asp-Ala-Ser-Ile-Ile-
Trp-Ala-Met-Met-Gln-Asn;
and
polypeptide VI:
(SEQ ID NO: 6)
Leu-Ser-Lys-Leu-Gly-Gly-Gly-Gly-Thr-Ser-Leu-Asp-
Ala-Ser-Ile-Ile-Trp-Ala-Met-Met-Gln-Asn-Gly-Gly-
Gly-Gly-Ser-Asp-Lys-Pro.

4. Use of the fused polypeptide with multifunctional activity according to claim 1 in the preparation of anti-fibrosis drugs.

5. Use of the fused polypeptide with multifunctional activity according to claim 1 in the preparation of antitumor drugs.

6. The use of the fused polypeptide with multifunctional activity in the preparation of anti-fibrosis drugs according to claim 4, wherein the fibrosis comprises pulmonary fibrosis, hepatic fibrosis, renal fibrosis, myocardial fibrosis, and skin fibrosis.

7. The use of the fused polypeptide with multifunctional activity in the preparation of antitumor drugs according to claim 5, wherein the tumors originated from human head and neck, brain, thyroid, esophagus, pancreas, liver, lung, stomach, breast, kidney, colon or rectum, ovary, cervix, uterus, prostate, melanoma, hemangioma, or sarcoma.

8. The use of the fused polypeptide with multifunctional activity in the preparation of anti-fibrosis drugs according to claim 4, wherein the fused polypeptide is a polypeptide or a pharmaceutically acceptable salt thereof, and a dosage form thereof is an injection, capsule, tablet, pill, nasal spray or aerosol of the polypeptide or the salt thereof.

9. The use of the fused polypeptide with multifunctional activity in the preparation of antitumor drugs according to claim 5, wherein the fused polypeptide is a polypeptide or a pharmaceutically acceptable salt thereof, and a dosage form thereof is an injection, capsule, tablet, pill, nasal spray or aerosol of the polypeptide or the salt thereof.


SEQUENCE LISTING (10 sequences)

SEQ ID NO: 1 - protein, 30 aa, Artificial Sequence
  synthesized
SDKPGGGGLSKLGGGGTSLDASIIWAMMQN

SEQ ID NO: 2 - protein, 30 aa, Artificial Sequence
  synthesized
SDKPGGGGTSLDASIIWAMMQNGGGGLSKL

SEQ ID NO: 3 - protein, 30 aa, Artificial Sequence
  synthesized
TSLDASIIWAMMQNGGGGSDKPGGGGLSKL

SEQ ID NO: 4 - protein, 30 aa, Artificial Sequence
  synthesized
TSLDASIIWAMMQNGGGGLSKLGGGGSDKP

SEQ ID NO: 5 - protein, 30 aa, Artificial Sequence
  synthesized
LSKLGGGGSDKPGGGGTSLDASIIWAMMQN

SEQ ID NO: 6 - protein, 30 aa, Artificial Sequence
  synthesized
LSKLGGGGTSLDASIIWAMMQNGGGGSDKP

SEQ ID NO: 7 - protein, 4 aa, Artificial Sequence
  synthesized
SDKP

SEQ ID NO: 8 - protein, 14 aa, Artificial Sequence
  synthesized
TSLDASIIWAMMQN

SEQ ID NO: 9 - protein, 4 aa, Artificial Sequence
  synthesized
LSKL

SEQ ID NO: 10 - protein, 4 aa, Artificial Sequence
  synthesized
GGGG